(19) United States
(12) Patent Application Publication
(10) Pub. No.: US 2010/0292454 A1
(43) Pub. Date: Nov. 18, 2010

(54) NEURONAL REGENERATION PROMOTING AGENT

(75) Inventors: Yuji Mishina, Cary, NC (US); Masahisa Yamada, Saitama (JP); Runa Araya, Kanagawa (JP); Haruo Kishida, Tokyo (JP); Kentaro Kogure, Shiga (JP); Hideyoshi Harashima, Hokkaido (JP)

Correspondence Address: GREENBLUM & BERNSTEIN, P.L.C., 1950 ROLAND CLARKE PLACE, RESTON, VA, 20191, US

(73) Assignees: Yuji Mishina, Apex, NC (US); RIKEN, Saitama (JP); NAT'L UNIVERSITY CORP. HOKKAIDO UNIVERSITY, Hokkaido (JP)

(21) Appl. No.: 11/993,575

(22) PCT Filed: Jun. 20, 2006
(86) PCT No.: PCT/JP2006/312284

§ 371 Date: Oct. 28, 2008

(30) Foreign Application Priority Data
Jun. 22, 2005 (JP) 2005-181699

(51) Int. Cl. C07H 21/02

(52) U.S. Cl. 536/24.5

(57) ABSTRACT

It is an object of the present invention to provide a novel neuronal regeneration promoting agent, particularly a neuronal regeneration promoting agent having an inhibitory effect on glial scar formation. The present invention provides a neuronal regeneration promoting agent which comprises an inhibitor of a bone morphogenetic protein type 1A receptor (BMPR1A) as an active ingredient.

### TECHNICAL FIELD

The present invention relates to a neuronal regeneration promoting agent. Specifically, the present invention relates to a neuronal regeneration promoting agent using an inhibitor of BMP receptor function.

### BACKGROUND ART

Neurons are tissue having no division potential in vivo, and thus, once damaged, the damage persists for a long period of time. In particular, the central nervous system including a brain and a spinal cord has no regenerative capacity. Therefore, effective therapeutic methods have not existed yet for traumatic injuries such as spinal cord injuries and neurodegenerative disorders such as Alzheimer's disease and Parkinson's disease. Meanwhile, peripheral nerves have regenerative capacity, however the regeneration thereof requires a time of several months to one year or more. Further, since the regeneration requires a long period of time, neurons may die out during that period, resulting in failure of the functional recovery. During this recovery period, neural cells called astrocytes change into proliferative cells called reactive astrocytes, forming a glial scar in the tissue. This serves as an obstacle to hinder the reprojection of regenerated neural axon. Accordingly, the development of a novel agent which can inhibit the glial scar formation has been desired.

The inhibition of the glial scar formation is essential for the establishment of neural tissue regeneration technologies. At present, there are technologies such as inhibition of cell proliferation by means of X-ray irradiation, and stem cell transplantation. The X-ray irradiation is limited to its exposure dose, and thus the application thereof to human is problematic in terms of technology. The stem cell transplantation is effective with respect to animal experiments, but has various problems when it comes to the application to human. Embryonic stem (ES) cells can be obtained by cloning a fertilized egg, however difficult obtainability of the fertilized egg is the problem. Further, the use of ES cells is ethically problematic especially for human. Moreover, undifferentiated mesenchymal stem cells (MSCs) of the bone marrow are present as adult stem cells substituting for ES cells. The MSC has been revealed to be differentiated into a bone, a cartilage, a muscle, an adipose, a blood vessel, and further a nerve. Also, MSC can be collected from the patient him/herself, and thus MSC is considered to be more clinically valuable than ES cell. However, MSC is problematic because only a trace amount thereof exists in the adult body, and particularly this tendency becomes more notable with age.

On the other hand, the bone morphogenetic protein has been named as an ectopic bone formation-inducing protein which exists in the bone matrix. The gene sequence thereof was elucidated, proving that the protein is a member of the TGF-β family. Moreover, there have been so far several reports on the receptor of bone morphogenetic proteins (Mishina Y. (2003) Function of bone morphogenetic protein signaling during mouse development. Front Biosci. 8, 855-869). The bone morphogenetic protein receptor genes have been cloned, and the nucleotide sequences thereof are registered in database (Mouse BMPR1A: NM_{—}009758; Rat BMPR1A: NM_{—}030849; and Human BMPR1A: NM_{—}004329).

### DISCLOSURE OF THE INVENTION

### Object to be Solved by the Invention

It is an objective to be solved by the present invention to provide a novel neuronal regeneration promoting agent, particularly a neuronal regeneration promoting agent having an inhibitory effect on glial scar formation.

### Means for Solving the Object

In order to solve the above problems, the inventors of the present invention have conducted intensive studies. As a result, they have found that glial scar formation can be specifically suppressed by inhibiting a bone morphogenetic protein type 1A receptor, and this has led to the completion of the present invention.

That is to say, according to the present invention, there is provided a neuronal regeneration promoting agent which comprises an inhibitor of a bone morphogenetic protein type 1 A receptor (BMPR1A) as an active ingredient.

Preferably, the inhibitor of a bone morphogenetic protein type 1A receptor (BMPR1A) is a substance which inhibits the expression of the bone morphogenetic protein type 1A receptor (BMPR1A).

Further preferably, the substance which inhibits the expression of the bone morphogenetic protein type 1A receptor (BMPR1A) is a substance which inhibits the expression of the bone morphogenetic protein type 1A receptor (BMPR1A) by RNAi.

Particularly preferably, the substance which inhibits the expression of the bone morphogenetic protein type 1A receptor (BMPR1A) by RNAi is an siRNA having the nucleotide sequence of SEQ ID NO: 1 in the sequence listing.

Preferably, the neuronal regeneration promoting agent of the present invention promotes the neuronal regeneration by inhibiting glial scar formation.

According to another aspect of the present invention, there is provided a method for promoting the neuronal regeneration, which comprises a step of administering a therapeutically effective dose of an inhibitor of a bone morphogenetic protein type 1A receptor (BMPR1A) to a mammal including a human.

According to yet another aspect of the present invention, there is provided a use of an inhibitor of a bone morphogenetic protein type 1A receptor (BMPR1A) for the production of a neuronal regeneration promoting agent.

### BEST MODE FOR CARRYING OUT THE INVENTION

Hereafter, embodiments of the present invention will be described.

A remarkable inhibitory effect on glial scar formation without causing any influence of side effects such as cell death on neurons, has been desired for the neural tissue. Bone morphogenetic proteins (BMPs) have been reported to have an effect of promoting neurite outgrowth, and thus have been desired to be developed into a substance having a high suppressing effect on the glial cell growth with less effect on neurons. The production of mice capable of destructing the bone morphogenetic protein type IA receptor (BMPR1A) gene in neurons and glial cells showed that the BMPR1A gene has such an effect. Thus, the inventors of the present invention found out that substances such as siRNA which specifically regulate the BMPR1A gene, and BMPR1A-specific antagonists have an inhibitory effect on the glial scar formation, and have shown examples using siRNA.

Moreover, it is also possible to incorporate siRNA which suppresses the BMPR1A gene expression in an “envelope-type nanostructure liposome” developed by the inventors of the present invention (refer to Journal of Controlled Release, Volume 98, Issue 2, 11 Aug. 2004, Pages 317-323, “Development of a non-viral multifunctional envelope-type nano device by a novel lipid film hydration method”; and Description in JP Patent Application No. 2005-61687). Gene transfection methods by means of this “envelope-type nanostructure liposome” are less cytotoxic as compared to conventional methods, and are capable of performing further highly efficient gene transfection. Since a gene can be transfected into growing cells alone, the target cells of the gene transfection can be limited. Accordingly, the glial scar formation can be inhibited by transfecting a gene into nuclei of growing glial cells.

Viral gene vectors are too risky to use in vivo because of their pathogenicity/immunogenicity. Therefore, nonviral gene vectors are desired. Conventional nonviral gene vectors (mainly lipoplex) have some problems, such as: 1) highly toxic due to the cationic lipids thereof; 2) easily degraded due to the uptake through an endocytosis pathway; and 3) provides nonuniform transfected cells. However, the vector used in the present invention (envelope-type nanostructure liposome) has a structure resembling envelope-type virus, the surface of which is arranged with arginine octamers that are multifunctional peptides. Such a unique structure provides the vector with advantages such as: 1) less toxic due to no use of cationic lipids; 2) hardly degraded and efficiently delivered to cytoplasm/nucleus due to the uptake through a nonendocytosis pathway; and 3) capable of transfecting a gene into 70% of cells or more, from other experiments, which are largely different from conventional nonviral gene vectors.

Specific examples of neurological disorders that can be applied with the neuronal regeneration promoting agent of the present invention include, but are not limited to, traumatic injuries such as spinal cord injuries and neurodegenerative disorders such as Alzheimer's disease and Parkinson's disease.

The bone morphogenetic protein type 1A receptor (BMPR1A) is a type of bone morphogenetic protein receptors, and the nucleotide sequences of the gene have already been reported (Mouse BMPR1A: NM_{—}009758; Rat BMPR1A: NM_{—}030849; and Human BMPR1A: NM_{—}004329). Nucleotide sequences of the gene of BMPR1A, a BMP receptor, of human, mouse, and rat are respectively shown in SEQ ID NOS:2 to 4.

The inhibitor of a bone morphogenetic protein type 1A receptor (BMPR1A) used in the present invention includes substances which inhibit the BMPR1A expression, substances which act on BMPR1A to inhibit the activity and the function of BMPR1A, and substances which inhibit the association between BMPR1A and BMP. The term “inhibition” used herein means suppression and/or reduction.

The substances which inhibit the BMPR1A expression include substances utilizing RNAi, an antisense method or a ribozyme method, and preferably siRNAs utilizing RNAi, although there is no particular limitation. The substances which act on BMPR1A to inhibit the activity and the function of BMPR1A include low molecular weight compounds and antibodies. As to the substances which inhibit the association between BMPR1A and BMP, there can be used low molecular weight compounds, antibodies, peptides, or the like.

Regarding the antibody, there can be used, for example, an antibody produced using a peptide having a full-length or a partial sequence of BMPR1A, as an immunogen. For example, a recombinant BMPR1A or the like may be used as the full-length BMPR1A. Such antibodies may be produced in accordance with a commonly used method. The antibody is preferably a monoclonal antibody. Examples of the peptide include peptides having a partial sequence of BMPR1A.

RNAi (RNA interference) refers to a phenomenon in which a double-strand RNA that has been transfected into a cell suppresses the expression of a gene having the same sequence.

Specific examples of substances which inhibit the BMPR1A expression by RNAi include siRNA and shRNA as described below.

The term siRNA is an abbreviation for short interfering RNA which refers to a double-strand RNA having a length of about 21 to 23 bp. The siRNA may be in any form as long as it is capable of inducing RNAi, examples of which may include: siRNAs obtained by a chemical synthesis, a biochemical synthesis, or an in vivo synthesis; and short double-strand RNAs of 10 by or more obtained by in vivo degradation of a double-strand RNA of about 40 by or more. The siRNA sequence and a partial mRNA sequence of BMPR1A preferably match 100%, but may not necessarily match 100%.

Preferably, the homologous region between the nucleotide sequence of siRNA and the nucleotide sequence of BMPR1A gene do not include the translation initiation region of BMPR1A gene. The homologous sequence is preferably apart from the translation initiation region of BMPR1A gene by 20 bp, and more preferably 70 bp. The homologous sequence may be, for example, a sequence in the vicinity of the 3′ terminal of BMPR1A gene.

As to the substance which inhibits the BMPR1A expression by RNAi, there may also be used ds RNA of about 40 by or more which generates siRNAs, and the like. For example, there may also be used RNA including a double-strand portion comprising a sequence having a homology with a part of the nucleic acid sequence of BMPR1A gene by about 70% or more, preferably 75% or more, more preferably 80% or more, yet more preferably 85% or more, even more preferably 90% or more, particularly preferably 95% or more, and most preferably 100%, and variants thereof. The sequence portion having a homology is normally at least 15 nucleotides or more, preferably about 19 nucleotides or more, more preferably at least 20 nucleotides or more, and yet more preferably 21 nucleotides or more.

As to the substance which inhibits the BMPR1A expression by RNAi, there may also be used shRNA (short hairpin RNA) comprising a short hairpin structure which projects at the 3′ terminal. The term shRNA refers to a molecule of about 20 bp or more, in which the single-strand RNA includes partially palindromic nucleotide sequences to thereby have a double-strand structure within the molecule, forming a hairpin-like structure. Moreover, the shRNA preferably has a projection at the 3′ terminal. There is no particular limitation on the length of the double-strand portion, although it is preferably 10 nucleotides or more, and more preferably 20 nucleotides or more. Here, the projecting 3′ terminal is preferably a DNA, more preferably a DNA of at least 2 or more nucleotides, and more preferably a DNA of 2 to 4 nucleotides.

The substance which inhibits the BMPR1A expression by RNAi may be artificially and chemically synthesized, and may also be produced through in vitro RNA synthesis using DNA of a hairpin structure in which a sense strand DNA sequence and an antisense strand DNA sequence are linked in opposite directions, with a T7 RNA polymerase. In the case of in vitro synthesis, antisense and sense RNAs can be synthesized from a template DNA using the T7 RNA polymerase and a T7 promoter. After in vitro annealing thereof, the resultant product is transfected into cells, so as to induce RNAi, suppressing the BMPR1A expression. For example, such transfection into cells can be carried out by a calcium phosphate method or a method using various transfection reagents (such as oligofectamine, lipofectamine, and lipofection).

As to the substance which inhibits the BMPR1A expression by RNAi, there may also used an expression vector which contains a nucleic acid sequence encoding the above siRNA or shRNA. Further, a cell containing the expression vector may also be used. There is no particular limitation on the types of the above expression vector and cell, although expression vectors and cells that are already in use as a medicament are preferred.

The administration route of the neuronal regeneration promoting agent of the present invention is not specifically limited, and any administration route may be taken such as oral administration, and parenteral administration (e.g., intravenous administration, intramuscular administration, subcutaneous administration, intradermal administration, transmucosal administration, intrarectal administration, intravaginal administration, local administration to the affected area, and skin administration). The suitable form of preparations for oral administration includes solid and liquid forms. The suitable form of preparation for parenteral administration includes forms such as an injectable solution, an solution for intravenous drip, a suppository, an external preparation, an ophthalmic solution, and a solution for nasal drops. The neuronal regeneration promoting agent of the present invention may be in the preparation form of a sustained-release agent. The neuronal regeneration promoting agent of the present invention may be mixed with a pharmaceutically acceptable additive, according to its preparation form, if necessary. Specific examples of such pharmaceutically acceptable additive include an excipient, a binder, a disintegrator, a lubricant, an antioxidant, a preservative, a stabilizer, an isotonizing agent, a coloring agent, a flavoring agent, a diluent, an emulsifier, a suspending agent, a solvent, a filler, an extending agent, a buffer agent, a delivery vehicle, a diluent, a carrier, an excipient, and/or a pharmaceutical adjuvant.

The neuronal regeneration promoting agent of the present invention in the form of a solid preparation for oral administration can be prepared, for example, in such a manner that a BMPR1A inhibitor serving as an active ingredient is added with an excipient, and, if necessary, further added with an additive for preparation such as a binder, a disintegrator, a lubricant, a coloring agent, or a flavoring agent, and the resultant mixture is then prepared to be in the form of tablets, granules, powder, or capsules by a usual method. The neuronal regeneration promoting agent of the present invention in the form of a liquid preparation for oral administration can be prepared, for example, in such a manner that a BMPR1A inhibitor serving as an active ingredient is added with one or more additives for preparation such as a flavoring agent, a stabilizer, and a preservative, and the resultant mixture is then prepared to be in the form of an internal liquid agent, a syrup, an elixir, and the like by a usual method.

A solvent that is used for prescribing the neuronal regeneration promoting agent of the present invention as a liquid preparation may be either aqueous or non-aqueous. Such a liquid preparation can be prepared by a publicly known method in the art. For example, an injectable solution can be prepared by dissolving in a solvent such as a physiological saline, a buffering solution e.g., PBS, and a sterilized water, then mechanically sterilizing through a filter or the like, and filling in a sterile container (such as an ampoule). This injectable solution may contain a commonly-used pharmaceutical carrier, if necessary. Moreover, an administration method using a noninvasive catheter, may also be employed. The carrier that can be used in the present invention includes a neutral buffered physiological saline, a serum albumin-containing physiological saline, and the like.

The way of transferring a gene such as an siRNA of a bone morphogenetic protein type 1 receptor and an siRNA expression vector is not particularly limited as long as an RNA encoding the siRNA of the bone morphogenetic protein type 1 receptor or the siRNA expression vector can be expressed in the neural tissue of an animal to be applied with the glial scar inhibitor. For example, a gene transfection using a viral vector or a liposome can be employed. Examples of the viral vector include animal viruses such as a retrovirus, a vaccinia virus, an adenovirus, and a semliki forest virus.

As a less toxic nonviral gene vector having a gene transfection efficiency that is as high as virus, it is desirable to use an “envelope-type nanostructure liposome” (refer to Journal of Controlled Release, Volume 98, Issue 2, 11 Aug. 2004, Pages 317-323, “Development of a non-viral multifunctional envelope-type nano device by a novel lipid film hydration method”; and Description in JP Patent Application No. 2005-61687). This method is considered to be capable of performing the gene transfection into a glial scar at the site of nerve injury safely and readily with a low cost.

Hereunder is a description of the envelope-type nanostructure liposome. The envelope-type nanostructure liposome preferably has a peptide comprising multiple consecutive arginine residues on the surface thereof.

As long as the envelope-type nanostructure liposome is a closed vesicle of a lipid bilayer membrane structure, there is no particular limitation on the number of lipid bilayers. The liposome may be either a multilayer vesicle (MLV) or monolayer vesicle such as an SUV (small monolayer vesicle), an LUV (large monolayer vesicle), and a GUV (giant monolayer vesicle).

The surface of a monolayer liposome means the external surface of the liposome membrane, while the surface of a multilayer liposome means the external surface of the outermost liposome membrane. The liposome may also have a peptide in a part other than the surface (such as the inner surface of a liposome membrane).

There is no particular limitation on the size of the liposome, although the diameter is preferably 50 to 800 nm, and more preferably 250 to 400 nm.

There is no particular limitation on the type of lipids making up the liposome membrane, and specific examples thereof include phosphatidylcholine (such as dioleoyl phosphatidylcholine, dilauroyl phosphatidylcholine, dimyristoyl phosphatidylcholine, dipalmitoyl phosphatidylcholine, and distearoyl phosphatidylcholine), phosphatidylglycerol (such as dioleoyl phosphatidylglycerol, dilauroyl phosphatidylglycerol, dimyristoyl phosphatidylglycerol, dipalmitoyl phosphatidylglycerol, and distearoyl phosphatidylglycerol), phosphatidylethanolamine (such as dioleoyl phosphatidylethanolamine, dilauroyl phosphatidylethanolamine, dimyristoyl phosphatidylethanolamine, dipalmitoyl phosphatidylethanolamine, and distearoyl phosphatidylethanolamine), phosphatidylserine, phosphatidylinositol, phosphatidic acid, cardiolipin, and other phospholipids, and hydrogenates thereof; and sphingomyelin, ganglioside, and other glycolipids, and one or more types thereof can be used. Phospholipids may be natural lipids derived from egg yolks, soy beans, or other animals or plants (such as yolk lecithin and soy lecithin), synthetic lipids, or semi-synthetic lipids. The lipid content in the liposome membrane is normally 70% to 100% (mole ratio), preferably 75% to 100% (mole ratio), and more preferably 80% to 100% (mole ratio) of the total amount of substances making up the liposome membrane.

In order to physically or chemically stabilize the liposome membrane or to adjust the fluidity of the liposome membrane, the liposome membrane may contain one or more types of: animal-derived sterols such as cholesterol, cholesterol succinic acid, lanosterol, dihydrolanosterol, desmosterol, and dihydrocholesterol; plant-derived sterols (phytosterols) such as stigmasterol, sitosterol, campesterol, and brassicasterol; microbiological sterols such zymosterol and ergosterol; the sugars such as glycerol and sucrose; and glycerin fatty acid esters such as triolein and trioctanoin. The content thereof is not particularly limited but is preferably 5% to 40% (mole ratio), and more preferably 10% to 30% (mole ratio) of the total lipids making up the liposome membrane.

The number of consecutive arginine residues in the peptide existing on the surface of the liposome is not particularly limited as long as it is more than one, but normally 4 to 20, preferably 6 to 12, and more preferably 7 to 10. The number of amino acid residues making up the aforementioned peptides not particularly limited as long as it is more than one, but normally 4 to 35, preferably 6 to 30, and more preferably 8 to 23. The aforementioned peptide may comprise any amino acid sequence at the C-terminal and/or N-terminal of the multiple consecutive arginine residues, but preferably all amino acid residues making up the peptide consist of arginine residues.

The amino acid sequence to be added to the C-terminal or N-terminal of the multiple consecutive arginine residues is preferably an amino acid sequence having rigidity (such as polyproline). Unlike polyethylene glycol which is soft in an irregular shape, polyproline is straight and maintains a certain hardness. Moreover, the amino acid residues included in the amino acid sequence to be added to the C-terminal or N-terminal of the multiple consecutive arginine residues are preferably not acidic amino acids. This is because acidic amino acids which carry a negative charge interact statically with arginine residues which carry a positive charge, potentially weakening the effect of the arginine residues.

The amount of the peptide existing on the surface of the liposome is normally 0.1% to 30% (mole ratio), preferably 1% to 25% (mole ratio), and more preferably 2% to 20% (mole ratio) of the total lipids making up the liposome membrane.

In the liposome used in the present invention, the liposome membrane may be composed of either a cationic lipid or a non-cationic lipid, or of both. However, since cationic lipids are cytotoxic, the amount of cationic lipids contained in the liposome membrane is preferably as small as possible in order to reduce the cytotoxicity of the liposome of the present invention, and the proportion of cationic lipids relative to total lipids making up the liposome membrane is preferably 0% to 40% (mole ratio) and more preferably 0% to 20% (mole ratio).

Examples of cationic lipids include DODAC (dioctadecyldimethylammonium chloride), DOTMA (N-(2,3-dioleyloxy)propyl-N,N,N-trimethylammonium), DDAB (didodecylammonium bromide), DOTAP (1,2-dioleyloxy-3-trimethylammonio propane), DC-Chol (3β-N-(N′,N′-dimethyl-aminoethane)-carbamol cholesterol), DMRIA (1,2-dimyristoyloxypropyl-3-dimethylhydroxyethyl ammonium), and DOSPA (2,3-dioleyloxy-N-[2(sperminecarboxamido)ethyl]-N,N-dimethyl-1-propanaminum trifluoroacetate).

The term non-cationic lipid means a neutral lipid or an anionic lipid. Examples of neutral lipids include diacylphosphatidylcholine, diacylphosphatidylethanolamine, cholesterol, ceramide, sphingomyelin, cephalin, and cerebroside. Examples of anionic lipids include cardiolipin, diacylphosphatidylserine, diacylphosphatidic acid, N-succinyl phosphatidylethanolamine(N-succinyl PE), phosphatidic acid, phosphatidylinositol, phosphatidylglycerol, phosphatidylethylene glycol, and cholesterol succinic acid.

An example of a preferred mode of the liposome is a liposome in which the peptide is modified with a hydrophobic group, the hydrophobic group is inserted into the lipid bilayer, and the peptide is exposed on the lipid bilayer. In this mode, “the peptide is exposed on the lipid bilayer” includes cases in which the peptide is exposed on either outer or inner surface of the lipid bilayer, or on both surfaces.

There is no particular limitation on the hydrophobic group as long as it can be inserted into the lipid bilayer. Examples of the hydrophobic group include saturated or unsaturated fatty acid groups such as a stearyl group, sterol residues such as a cholesterol residue, phospholipid residues, glycolipid residues, long-chain aliphatic alcohol residues (such as phosphatidylethanolamine residues), polyoxypropylene alkyl residues, and glycerin fatty acid ester residues. Of these, a fatty acid group of 10 to 20 carbon atoms (such as a palmitoyl group, an oleoyl group, a stearyl group, and an arachidoyl group) is particularly preferred.

The liposome can be produced by a publicly known method such as a hydration method, a ultrasonic treatment method, an ethanol injection method, an ether injection method, a reverse-phase evaporation method, a surfactant method, and a freezing and thawing method.

A production example using the hydration method is shown below.

A lipid which is a component of the liposome membrane is dissolved in an organic solvent together with the peptide modified with a hydrophobic group, and then the organic solvent is removed by evaporation to obtain a lipid membrane. Examples of the organic solvent used herein include: hydrocarbons such as pentane, hexane, heptane, and cyclohexane; halogenated hydrocarbons such as methylene chloride and chloroform; aromatic hydrocarbons such as benzene and toluene; lower alcohols such as methanol and ethanol; esters such as methyl acetate and ethyl acetate; and ketones such as acetone, which may be used either singularly or in combination of two or more types thereof. Next, the lipid membrane is hydrated, and then subjected to agitation or ultrasonic treatment to produce a liposome having the peptide on its surface.

Another production example using the hydration method is described below.

A lipid which is a component of the lipid bilayer is dissolved in an organic solvent, and then the organic solvent is removed by evaporation to obtain a lipid membrane. This lipid membrane is hydrated, and then is subjected to agitation or ultrasonic treatment to produce a liposome. Next, the peptide modified with a hydrophobic group is added to the external solution of the liposome to thereby introduce the peptide into the surface of the liposome.

Liposomes having a fixed particle size distribution can be obtained by passing the liposomes through a filter of a predetermined pore size. Moreover, multilayer liposomes can be converted into monolayer liposomes or monolayer liposomes can be converted into multilayer liposomes according to a publicly known method.

The substance which inhibits the BMPR1A expression by RNAi can be directly injected into an organ, tissue, or the like in vivo.

The dose of the neuronal regeneration promoting agent of the present invention can be determined by those skilled in the art with a consideration of the purpose of administration, the degree of seriousness of the disease, the age, weight, gender, and previous history of the patient, and the type of the substance which inhibits the BMPR1A expression by RNAi, serving as an active ingredient. The dose of the neuronal regeneration promoting agent of the present invention is, for example, in cases where the active ingredient is a substance which inhibits the BMPR1A expression by RNAi, about 0.1 ng to about 100 mg/kg, and preferably about 1 ng to about 10 mg per adult, as the amount of the active ingredient; and in cases where the promoting agent is to be administered in the form of a viral vector or a nonviral vector, normally 0.0001 to 100 mg, preferably 0.001 to 10 mg, and more preferably 0.01 to 1 mg.

The frequency of administration of the neuronal regeneration promoting agent of the present invention may be for example, once a day to once per several months. If a substance which inhibits the BMPR1A expression by RNAi is used, its effect is typically exerted for one to three days after the administration, and thus the frequency of administration is preferably everyday to every third day. If an expression vector is used, the administration might be appropriately about once a week in some cases.

### EXAMPLES

### Example 1

### Experimental Method

A total of 2×10⁵ mouse primary cultured astrocytes were plated on a collagen-coated 6-well plate. On the next day, these cells were transfected using Lipofectamin™ 2000 reagent (Invitrogen) in accordance with an appended instruction. In brief, 80 pmol of either BMPR1A siRNA (AAGGGCAGAAUCUAGAUAGUA: SEQ ID NO:1) (corresponding to 65th-85th base sequence of SEQ ID NO:3) or Lamin A/C siRNA (Qiagen) was mixed with 100 μl of Opti-MEM (GIBCO), to which 4 μl of Lipofectamin™ 2000 reagent was added. After 20 minutes incubation, the siRNA-lifectamine complex was applied to respective wells along with 800 μl of Opti-MEM.

After 3 days post-transfection, the total RNA was extracted from the confluently cultured cells on the 6-well plate using an RNeasy Mini Kit (Qiagen). Then, Taq Man real time RT-PCR was performed using 2 μg of the total RNA. Table 1 shows average values of four samples.

TABLE 1

| | | BMPR1A/ | Sample/ | AVG | SEM |
|---|---|---|---|---|---|
| | | GAPDH | control (%) | (%) | (%) |
| non-treat | control #1 | 1.65 | 92.17 | 99.99 | 5.35 |
| aastrocytes | control #2 | 1.85 | 103.70 | | |
| | control #3 | 1.62 | 90.65 | | |
| | control #4 | 2.03 | 113.47 | | |
| siRMPR1A | sample #1 | 0.29 | 16.46 | 16.25 | 2.29 |
| | sample #2 | 0.39 | 22.30 | | |
| | sample #3 | 0.26 | 14.96 | | |
| | sample #4 | 0.20 | 11.25 | | |

The results of Table 1 showed that BMPR1A siRNA suppressed the BMPR1A mRNA expression level to about 16% in the mouse astrocyte primary culture system.

### Example 2

### Experimental Method

Gene transfection was performed using an siRNA having an suppressing effect on the BMPR1A receptor gene expression, into confluently cultured astrocytes, by means of lipofection. After 3 days, the astrocytes were scratched with a needle to perform an experiment on glial scar formation. The inhibitory activity on the astrocyte growth was measured by fluorescent staining with cell nuclear staining (blue), bromo-2-deoxyuridine (BrdU; red) which indicates growing cells, and GFP-labeled siRNA (green). Experimental method is shown in (1) to (4) as below.

### (1) Culture of Primary Mouse Glial Cells

Primary astroglial cell culture of isolated cortical brain cell was prepared from the brain of an E17 mouse (ICR: Japan SLC). The cortical brain-derived tissue pieces were incubated in Ca²⁺- and Mg²⁺- free PBS (5 ml) containing 0.25% trypsin (GIBCO) and DNase I (100 units; Boehringer Mannheim) at 37° C. for 15 minutes. The cells were mechanically isolated by pipetting, and then were resuspended in DMEM containing 10% FBS. The isolated cells were placed in a polyethyleneimine-coated flask, and cultured for 7 days. Then, the cells were again plated on a polyethyleneimine-coated dish at a final cell density of 3×10⁴ cells/cm², and were cultured in DMEM containing 20% FBS for another 6 days.

(2) Transfection of siRNA

A total of 5×10⁴ cells were plated on a collagen-coated 48-well plate. On the next day, these cells were transfected using Lipofectamin™ 2000 reagent (Invitrogen) in accordance with an appended instruction. In brief, 10 pmol of BLOCK-iT™ Fluorescent Oligo (Invitrogen) and 20 pmol of either BMPR1A siRNA (AAGGGCAGAAUCUAGAUAGUA: SEQ ID NO:1) or Lamin A/C siRNA (Qiagen) were mixed with 25 μl of Opti-MEM (GIBCO), to which 0.5 μl of Lipofectamin™ 2000 reagent was added, followed by 20 minutes incubation. The siRNA-lifectamine complex was applied to respective wells along with 200 μl of Opti-MEM.

### (3) Cell Growth Assay

After 3 days post-transfection, confluently cultured cells on the 48-well plate were scratched using a 0.1% (V/V) standard reagent (labelling reagent) (cell proliferation KIT, Amersham Biosciences) in DMEM containing 2.5% FBS, to cause impairment. On the next day, the cells were fixed to perform immunostaining.

### (4) Immunostaining

For the immunocytochemical analysis, the cells were washed with PBS and fixed with 4% paraformaldehyde, which were subjected to 30 mins treatment with 90% ethanol and 5% acetic acid, and subsequent 30 mins incubation in 2% H₂O₂ (in methanol). The resultant cells were blocked with PBS containing 5% normal goat serum (Vector Laboratories Inc.) and 0.01% Triton, and then were incubated with an anti-bromodeoxyuridine antibody (mouse monoclonal antibody, RPN202, Amersham Biosciences). In the present experiment, anti-mouse antibodies (molecular probes) bound to Alexa 546 were used as the secondary antibody. After washing, the cells were exposed to Hoechst 33258, and analyzed using a non-confocal fluorescence microscope (1×71, Olympus).

### Experimental Results

The experimental results are shown in FIG. 1. From the results of FIG. 1, the transfection of a BMPR1A-specific siRNA lowered the ratio of bromodeoxyuridine-positive cells, showing that the astrocyte growth was inhibited. From the above experimental results, siRNA which inhibits the BMPR1A gene expression was proven to significantly inhibit the glial scar formation in vitro.

### BRIEF DESCRIPTION OF THE DRAWINGS

FIG. 1 shows the measurement results of inhibitory activity on the astrocyte growth when transfected with either a nonspecific siRNA or a BMPR1A-specific siRNA.

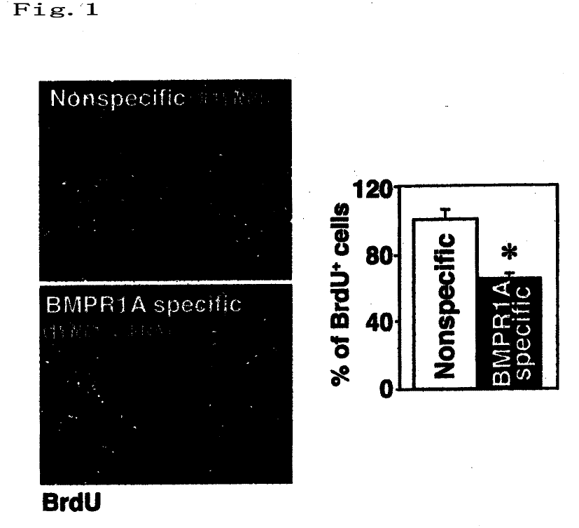

### INDUSTRIAL APPLICABILITY

The present invention provides a novel neuronal regeneration promoting agent having an inhibitory effect on glial scar formation.

1. A neuronal regeneration promoting agent which comprises an inhibitor of a bone morphogenetic protein type 1A receptor (BMPR1A) as an active ingredient.

2. The neuronal regeneration promoting agent of claim 1 wherein the inhibitor of a bone morphogenetic protein type 1A receptor (BMPR1A) is a substance which inhibits the expression of the bone morphogenetic protein type 1A receptor (BMPR1A).

3. The neuronal regeneration promoting agent of claim 1 wherein the substance which inhibits the expression of the bone morphogenetic protein type 1A receptor (BMPR1A) is a substance which inhibits the expression of the bone morphogenetic protein type 1A receptor (BMPR1A) by RNAi.

4. The neuronal regeneration promoting agent of claim 3 wherein the substance which inhibits the expression of the bone morphogenetic protein type 1A receptor (BMPR1A) by RNAi is an siRNA having the nucleotide sequence of SEQ ID NO: 1 in the sequence listing.

5. The neuronal regeneration promoting agent of claim 1 which promotes the neuronal regeneration by inhibiting glial scar formation.

6. The neuronal regeneration promoting agent of claim 2 wherein the substance which inhibits the expression of the bone morphogenetic protein type 1A receptor (BMPR1A) is a substance which inhibits the expression of the bone morphogenetic protein type 1A receptor (BMPR1A) by RNAi.

7. The neuronal regeneration promoting agent of claim 6 wherein the substance which inhibits the expression of the bone morphogenetic protein type 1A receptor (BMPR1A) by RNAi is an siRNA having the nucleotide sequence of SEQ ID NO: 1 in the sequence listing.

8. The neuronal regeneration promoting agent of claim 2 which promotes the neuronal regeneration by inhibiting glial scar formation.

9. The neuronal regeneration promoting agent of claim 3 which promotes the neuronal regeneration by inhibiting glial scar formation.

10. The neuronal regeneration promoting agent of claim 4 which promotes the neuronal regeneration by inhibiting glial scar formation.

11. The neuronal regeneration promoting agent of claim 5 which promotes the neuronal regeneration by inhibiting glial scar formation.

12. The neuronal regeneration promoting agent of claim 6 which promotes the neuronal regeneration by inhibiting glial scar formation.

13. The neuronal regeneration promoting agent of claim 7 which promotes the neuronal regeneration by inhibiting glial scar formation.


SEQUENCE LISTING (4 sequences)

SEQ ID NO: 1 - RNA, 21 nt, Artificial Sequence
  Description of Artificial Sequence Synthetic transcribed oligonucleotide
aagggcagaaucuagauagua

SEQ ID NO: 2 - DNA, 1599 bp, Homo sapiens
atgactcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctga

SEQ ID NO: 3 - DNA, 1599 bp, Mus musculus
atgactcagctatacacttacatcagattactgggagcctgtctgttcatcatttctcatgttcaagggcagaatctagatagtatgctccatggcactggtatgaaatcagacttggaccagaagaagccagaaaatggagtgactttagcaccagaggataccttgcctttcttaaagtgctattgctcaggacactgcccagatgatgctattaataacacatgcataactaatggccattgctttgccattatagaagaagatgatcagggagaaaccacattaacttctgggtgtatgaagtatgaaggctctgattttcaatgcaaggattcaccgaaagcccagctacgcaggacaatagaatgttgtcggaccaatttgtgcaaccagtatttgcagcctacactgccccctgttgttataggtccgttctttgatggcagcatccgatggctggttgtgctcatttccatggctgtctgtatagttgctatgatcatcttctccagctgcttttgctataagcattattgtaagagtatctcaagcaggggtcgttacaaccgtgatttggaacaggatgaagcatttattccagtaggagaatcattgaaagacctgattgaccagtcccaaagctctgggagtggatctggattgcctttattggttcagcgaactattgccaaacagattcagatggttcggcaggttggtaaaggccgctatggagaagtatggatgggtaaatggcgtggtgaaaaagtggctgtcaaagtgttttttaccactgaagaagctagctggtttagagaaacagaaatctaccagacggtgttaatgcgtcatgaaaatatacttggttttatagctgcagacattaaaggcactggttcctggactcagctgtatttgattactgattaccatgaaaatggatctctctatgacttcctgaaatgtgccacactagacaccagagccctactcaagttagcttattctgctgcttgtggtctgtgccacctccacacagaaatttatggtacccaagggaagcctgcaattgctcatcgagacctgaagagcaaaaacatccttattaagaaaaatggaagttgctgtattgctgacctgggcctagctgttaaattcaacagtgatacaaatgaagttgacatacccttgaataccagggtgggcaccaagcggtacatggctccagaagtgctggatgaaagcctgaataaaaaccatttccagccctacatcatggctgacatctatagctttggtttgatcatttgggaaatggctcgtcgttgtattacaggaggaatcgtggaggaatatcaattaccatattacaacatggtgcccagtgacccatcctatgaggacatgcgtgaggttgtgtgtgtgaaacgcttgcggccaatcgtgtctaaccgctggaacagcgatgaatgtcttcgagcagttttgaagctaatgtcagaatgttgggcccataatccagcctccagactcacagctttgagaatcaagaagacacttgcaaaaatggttgaatcccaggatgtaaagatttga

SEQ ID NO: 4 - DNA, 1599 bp, Rattus norvegicus
atgactcagctatacacttacatcagattactgggagcctgtctgttcatcatttctcatgttcaagggcagaatctagatagtatgctccatggtactggtatgaaatcagacgtggaccagaagaagccggaaaatggagtgacgttagcaccagaggacaccttacctttcttaaaatgctattgctcaggacactgcccagatgacgctattaataacacatgcataactaatggccattgctttgccattatagaagaagatgatcagggagaaaccacgttaacttctgggtgtatgaagtatgaaggctctgattttcaatgcaaggattcaccaaaagcccagctacgcaggacaatagaatgttgtcggaccaatttgtgcaaccaatatttgcagcctacactgccccctgtcgttataggcccattctttgatggcagcgtccgatggctggctgtgctcatctctatggctgtctgtattgtcgccatgatcgtcttctccagctgcttctgttacaaacattactgtaagagtatctcaagcagaggtcgttacaaccgtgacttggaacaggatgaagcatttattccagtaggagaatcactgaaagacctgattgaccagtcacaaagctctggtagtggatctggattacctttattggttcagcgaactattgccaaacagattcagatggttcggcaggttggtaaaggccggtatggagaagtatggatgggtaaatggcgtggtgaaaaagtggctgtcaaagtattttttaccactgaagaagctagctggtttagagaaacagaaatctaccagacggtgttaatgcgtcatgaaaatatacttggttttatagctgcagacattaaaggcaccggttcctggactcagctgtatttgattactgattaccatgagaatgggtctctctatgacttcctgaaatgtgccaccctggacaccagagccctactcaagttagcttattctgctgcctgtggtctgtgccacctccacacagaaatttatggcacgcaaggcaagcctgcaattgctcatcgagacctgaagagcaaaaacatccttattaagaaaaatggtagttgctgtattgctgacctgggcctagctgttaaattcaacagtgacacaaatgaagttgacatacccttgaacaccagggtgggcaccaggcggtacatggctccagaagtgctggacgagagcctgagtaaaaaccatttccagccctacatcatggctgacatctacagctttggtttgatcatttgggagatggcccgtcgctgtattacaggaggaatcgtggaggaatatcaattaccatattacaacatggtgcctagtgacccatcttatgaagacatgcgtgaggtcgtgtgtgtgaaacgcttgcggccaatcgtctctaaccgctggaacagtgatgaatgtcttcgagccgttttgaagctgatgtcagaatgctgggcccataatccagcatccagactcacagctttgagaatcaagaagacgctcgcaaagatggttgaatcccaggatgtaaagatttga